SEQUENCE LISTING (5 sequences)

SEQ ID NO: 1 - protein, 25 aa, Artificial Sequence
  Synthetic poly(Glu,Tyr) peptide
EEEEYEEEEYEEEEYEEEEYEEEEY

SEQ ID NO: 2 - DNA, 75 bp, Artificial Sequence
  Synthetic DNA encoding poly(Glu,Tyr) peptide
gaggaggaggagtacgaagaagaagaatatgaagaagaagaatatgaagaagaagaatatgaggaggaagagtac

SEQ ID NO: 3 - DNA, 25 bp, Artificial Sequence
  Synthetic oligonucleotide based on nucleotide sequence of poly(Glu,Tyr) peptide
ccggggatccgaggaggaggagtac

SEQ ID NO: 4 - DNA, 27 bp, Artificial Sequence
  Synthetic oligonucleotide based on nucleotide sequence of poly(Glu,Tyr) peptide
cccggggaattcgtactcttcttcctc

SEQ ID NO: 5 - protein, 263 aa, Artificial Sequence
  Synthetic GST-poly(Glu,Tyr) fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEEEEYEEEEYEEEEYEEEEYEEEEYEFPGRLERPHRD

(19) United States
(12) Patent Application Publication
(10) Pub. No.: US 2009/0098582 A1
(43) Pub. Date: Apr. 16, 2009

(54) METHOD FOR DETERMINING RISK OF CANCER RELAPSE AND COMPUTER PROGRAM PRODUCT

(75) Inventors: Tomoko OYAMA, Akashi-shi (JP); Michitaka Notoya, Kobe-shi (JP); Tomokazu Yoshida, Kobe-shi (JP); Hideki Ishihara, Miki-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/249,417

(22) Filed: Oct. 10, 2008

(30) Foreign Application Priority Data
Oct. 10, 2007 (JP) 2007-264613

(51) Int. Cl. G01N 33/53; G06F 19/00

(52) U.S. Cl. 435/7.72; 702/19

(57) ABSTRACT

The invention provides a method for determining a risk of cancer relapse comprising a step of determining the risk of cancer relapse on the basis of a comprehensive activity value of a transmembrane tyrosine kinase of a tumor cell, as well as a computer program product for determining a risk of a cancer relapse.

### FIELD OF THE INVENTION

The present invention relates to a method for determining a risk of cancer relapse and a computer program product. More specifically, the present invention relates to a method for determining a risk of cancer relapse on the basis of an analytical result obtained by analyzing a molecule of a cell contained in a tumor tissue collected from a cancer patient as well as a computer program product.

### BACKGROUND

In recent years, a risk of cancer relapse has been clinically determined on the basis of various parameters. For example, it has been proposed that a risk of relapse of an early breast cancer is determined on the basis of the expression level of HER2 that is one kind of growth factor receptor having a tyrosine kinase activity (Joensuu H., et al., Clinical Cancer Research, vol. 9, 2003, 923-930). Further, a method of determining a risk of cancer relapse on the basis of the ratio of activity value versus expression level of a cyclin-dependent kinase has been proposed (U.S. Patent Application Publication No. 2007-231837).

Clinical determination of a risk of cancer relapse can influence postoperative therapeutic strategy and the like, and should thus be accurately and stably conducted. Accordingly, it has been examined to determine a risk of cancer relapse by using parameters related to malignant transformation of cells, other than the parameters mentioned above.

### SUMMARY

The scope of the present invention is defined solely by the appended claims, and is not affected to any degree by the statements within this summary.

The present invention has made in view of such circumstances. An object of the present invention is to provide a method that can determine a risk of cancer relapse by using a new parameter related to malignant transformation of cells.

Another object of the present invention is to provide a computer program product for determining a risk of cancer relapse by using the determining method described above.

The present inventors have found that a risk of cancer relapse can be determined by using the above parameter on the basis of a comprehensive activity value of a transmembrane tyrosine kinase, and the present invention has been thereby completed.

According to the present invention, a risk of cancer relapse can be determined by using a new parameter that is a comprehensive activity value of a transmembrane tyrosine kinase, which reflects the activity value of various types of transmembrane tyrosine kinases occurring in tumor cells.

### BRIEF DESCRIPTION OF THE DRAWINGS

FIG. 1 is a schematic view of a computer system for performing the method in the present embodiment;

FIG. 2 is a judgment flow based on the determining method in a first embodiment;

FIG. 3 shows a result of detection of the phosphorylation, with a transmembrane tyrosine kinase, of a GST-poly(Glu, Tyr) substrate prepared in the present embodiment;

FIG. 4 is a graph showing the relationship between a patient group (abscissa) and a comprehensive activity value (ordinate) of a transmembrane tyrosine kinase; and

FIG. 5 is a graph showing the relationship between a CDK specific activity ratio (abscissa) and a comprehensive activity value (ordinate) of a transmembrane tyrosine kinase.

### DETAILED DESCRIPTION OF THE EMBODIMENT

The determining method in the first embodiment of the present invention is a method for determining a risk of cancer relapse on the basis of a comprehensive activity value of a transmembrane tyrosine kinase occurring in a tumor cell.

The cancer to which the determining method is applied includes hematopoietic organ-derived malignant tumors, epidermal cell-derived carcinomas, sarcomas, and the like. The hematopoietic organ-derived malignant tumors can be exemplified by leukemia, malignant lymphoma, and the like. The carcinomas can be exemplified by breast cancer, stomach cancer, colon cancer, esophageal cancer, prostate cancer, and the like. The sarcomas can be exemplified by osteosarcoma, soft-tissue sarcoma, and the like.

The cell used in determination may be a cell contained in a tumor tissue collected from a cancer patient. The cell used in determination may be any cell contained in a tumor tissue of an initial cancer, an early cancer or an advanced cancer. For example, the cell used in determination of breast cancer is preferably a cell of a malignant tumor in “stage I” to “stage IIIA” in a stage classification. A major characteristic of the present invention is that a risk of relapse of not only an early cancer but also an advanced cancer can be determined.

The stage classification is a classification showing a malignancy of the malignant tumor. For example, the breast cancer is classified into stages I, IIA, IIB, IIIA, IIIB and IV in the order of increasing malignancy. The stage classification is based on TNM classification. The TNM classification is a stage classification of the malignant tumor according to the Union Internationale Contra le Cancer (UICC, International Union Against Cancer). “T” denotes a size of a primary tumor and ranges from T0 (primary lesion cannot be confirmed) to T4 (tumor is exposed out of body). “N” denotes a degree of invasion into lymphonodus in vicinity and ranges from N0 (no metastasis into lymphonodus) to N3 (metastasis into lymphonodus in the vicinity of a median part of a body (lymphonodus in vicinity of breast bone) is suspected). “M” denotes the presence or absence of remote metastasis and ranges from M0 (no remote metastasis) to M1 (remote metastasis is confirmed).

The tumor tissue includes, for example, tumor tissues excised by surgical operation and tumor tissues collected by biopsy.

According to the determining method in the present embodiment, a risk of cancer relapse (relapse likelihood) can be determined.

Relapse refers to a case where a tumor is excised from the living body, and after a predetermined time, a malignant tumor reappears in the same site, and also to a case where a cancerous cell is separated from a primary tumor and carried into a distant tissue, to proliferate autonomically therein. Whether a relapse occurs or not depends on the growth potential, viability, and migration capability of tumor cells.

The comprehensive activity value of a transmembrane tyrosine kinase refers to a comprehensively measured activity value of various transmembrane tyrosine kinases occurring in cell membranes of tumor cells, without isolating and purifying the transmembrane tyrosine kinases. This comprehensive activity value can be measured for example by using either a mixture of substrates that are highly specific for predetermined transmembrane tyrosine kinases or a universal substrate for various types of transmembrane tyrosine kinases.

The comprehensive activity value of a transmembrane tyrosine kinase reflects an activity value of various transmembrane tyrosine kinases occurring in cell membranes of tumor cells. The determination result of a risk of cancer relapse obtained based on the comprehensive activity value of transmembrane tyrosine kinases serves as very useful information in examining therapeutic strategies and the like after cancer surgery.

The transmembrane tyrosine kinase refers to a transmembrane tyrosine kinase occurring in a cell membrane. The transmembrane tyrosine kinase plays an important role in cell growth, cell survival, cell differentiation and angiogenesis. Many transmembrane tyrosine kinases are involved in malignant transformation of cells. It is known that an abnormality in the expression level and enzyme activity of the transmembrane tyrosine kinase causes malignant transformation of cells. Specific transmembrane tyrosine kinases include growth factor receptors such as an insulin-like growth factor receptor (IGFR), a platelet-derived growth factor receptor (PDGFR), a human epithelial growth factor receptor (HER), and a vascular endothelial growth factor receptor (VEGFR). The HER family includes HER1, HER2, HER3 and HER4.

The transmembrane tyrosine kinase is recovered from a cell membrane of a cell. The cell may be a cell contained in a biological sample collected from a living body or a cultured cell in a cell line established from a cell collected from a living body. Specifically, the cell may be a tumor cell. The tumor cell may be a tumor cell contained in a biological sample (tumor tissue) collected from a cancer patient or a cultured cell in a cell line established from a tumor cell collected from a cancer patient.

The method of recovering a transmembrane tyrosine kinase is not particularly limited as long as a sample containing various types of transmembrane tyrosine kinases can be prepared by separating the cytoplasm from a cell. A transmembrane tyrosine kinase-containing sample can be prepared for example by the following:
(1) a step of crushing cell membranes of cells in a suitable buffer (referred to hereinafter as a homogenizing reagent);
(2) a step of separating the resulting liquid by centrifugation into a supernatant and precipitates;
(3) a step of removing the supernatant;
(4) a step of mixing the precipitates with a surfactant-containing solution (referred to hereinafter as a lysing treatment solution); and
(5) a step of separating the resulting mixture by centrifugation into a supernatant and precipitates;
(6) whereby the supernatant is provided as a transmembrane tyrosine kinase-containing sample.
   The supernatant separated in step (2) contains proteins and the like derived from the cytoplasm. The precipitates separated in step (2) contain fragments of cell membrane maintaining various transmembrane tyrosine kinases. The supernatant separated in step (5) contains cell membranes containing various transmembrane tyrosine kinases formed into micelles by a surfactant. The precipitates separated in step (5) contain insoluble proteins, DNAs, and the like. The transmembrane tyrosine kinases contained in the sample prepared by the method described above occur in such a state that they penetrate cell membranes.

It is known that transmembrane tyrosine kinases, upon having a ligand bound thereto, forms a homodimer or a heterodimer. The sample prepared by the method described above contains transmembrane tyrosine kinases retaining their steric structure in such a state as to be able to form a homodimer or a heterodimer. Accordingly, a risk of cancer relapse can be determined more accurately by using the sample prepared by the method described above.

When cells are crushed, the homogenizing reagent is used to prevent the denaturation of transmembrane tyrosine kinases. The pH of the homogenizing reagent is not particularly limited as long as transmembrane tyrosine kinases can be recovered in a stable state without denaturation and inactivation of the transmembrane tyrosine kinases. The pH is specifically in the range of pH 4.0 to 9.0. The pH is preferably in the range of pH 4.5 to 8.5. The pH is particularly preferably in the range of 5.0 to 8.0.

The homogenizing reagent preferably contains a buffer. The buffer includes, for example, a phosphate buffer, an acetate buffer, a citrate buffer, MOPS (3-morpholinopropanesulfonic acid), HEPES (2-[4-(2-hydroxyethyl)-1-piperazinyl]ethanesulfonic acid), Tris (tris(hydroxymethyl)aminomethane), and Tricine (N-tris(hydroxymethyl)methylglycine).

The lysing treatment solution is preferably a solution also containing the buffer described above and having the same pH as in the homogenizing reagent. A protease inhibitor, a phosphatase inhibitor, and a reagent for preventing the oxidation of SH group (referred to hereinafter as SH group stabilizer) may be added to the homogenizing reagent and/or the lysing treatment solution.

The method of crushing the cell membrane is not particularly limited as long as the cell membrane can be fragmented. The method includes, for example, suctioning and discharging with a pipette, stirring with a vortex mixer, crushing with a blender, pressurization with a pestle, and sonication with an ultrasonicator.

The surfactant contained in the lysing treatment solution is used in lysing the fragmented cell membranes (formation of micelles). The surfactant used herein is preferably one which does not decompose or denature the transmembrane tyrosine kinases contained in cell membranes. An electrically charged surfactant may bind to a transmembrane tyrosine kinase to change a steric structure of the transmembrane tyrosine kinase. Accordingly, a nonionic surfactant that does not substantially bind to a transmembrane tyrosine kinase is preferably used. The nonionic surfactants include, for example, those having, as a fundamental structure, dodecyl ether, cetyl ether, stearyl ether, p-t-octyl phenyl ether, or the like. Specific examples include Nonidet P-40 (registered trademark) (NP-40, Shell International Petroleum Company Limited), Triton-X (registered trade mark) (Union Carbide Chemicals and Plastics Inc.), Tween (registered trade mark) (ICI Americas Inc.), Brij (registered trade mark) (ICI Americas Inc.), Emulgen (registered trade mark) (Kao Corporation) and the like. The concentration of the surfactant in the lysing treatment solution is preferably 0.05 to 5%, more preferably 0.1 to 3%, still more preferably 0.1 to 0.11%.

The protease inhibitor can be used to prevent transmembrane tyrosine kinases from being decomposed with proteases contained in cells. The protease inhibitor includes, for example, metalloprotease inhibitors such as EDTA and EGTA, serine protease inhibitors such as PMSF, trypsin inhibitor and chymotrypsin, and cysteine protease inhibitors such as iodoacetamide and E-64. These protease inhibitors may be used alone or as a mixture of two or more thereof. A commercial product such as a protease inhibitor cocktail (Sigma) in which a plurality of protease inhibitors has been mixed can also be used.

The phosphatase inhibitor can be used to prevent the enzyme activity of transmembrane tyrosine kinases from being deteriorated with phosphatases contained in cells. The phosphatase inhibitor includes, for example, sodium o-vanadate (Na₃VO₄), sodium fluoride (NaF), and okadaic acid. The phosphatase inhibitors may be used alone or as a mixture of two or more thereof.

The SH group stabilizer can be used to prevent the inactivation of a transmembrane tyrosine kinase. An SH group contained in the enzyme easily forms a disulfide by oxidization. Disulfide formation causes a change in the structure of the enzyme. Accordingly, disulfide formation may cause inactivation of the enzyme. Oxidation of SH group can be prevented by an SH group-containing reagent. The SH group stabilizer includes, for example, dithiothreitol (DTT), 2-mercaptoethanol, glutathione, cysteine, homocysteine, coenzyme A, and dihydrolipoic acid. The concentration of the SH group stabilizer in the homogenizing reagent and/or the lysing treatment solution may be suitably adjusted depending on the type of the SH group stabilizer. For example, when DTT is used as the SH group stabilizer, its concentration is 0.05 to 2 mM, preferably 0.07 to 1.7 mM, and particularly preferably 0.1 to 1.5 mM. When2-mercaptoethanol is used as the SH group stabilizer, its concentration is 0.1 to 1.5 mM, preferably 0.3 to 13 mM, and particularly preferably 0.5 to 12 mM.

In the present embodiment, the cytoplasm is separated from a cell to prepare a sample containing plural types of transmembrane tyrosine kinases, and the comprehensive activity value of transmembrane tyrosine kinases in this sample is measured.

For measuring the comprehensive activity value of transmembrane tyrosine kinases, substrates for at least 2 types of transmembrane tyrosine kinases are utilized. Specifically, transmembrane tyrosine kinases contained in a sample are contained with substrates for at least 2 types of transmembrane tyrosine kinases. By this contact, the substrates are phosphorylated by the activity of transmembrane tyrosine kinases in a sample. Then, the phosphorylated substrates are detected, and the comprehensive activity value of the transmembrane tyrosine kinases can be measured on the basis of the obtained detection result. Preferably, a sample containing transmembrane tyrosine kinases, substrates for at least 2 types of transmembrane tyrosine kinases, and a phosphate group donor are mixed and contacted with one another to detect the substrates phosphorylated by the activity of the tyrosine kinases. The transmembrane tyrosine kinase is activated by autophosphorylation. However, the transmembrane tyrosine kinase even when autophosphorylated does not always phosphorylate the substrate. Accordingly, the activity value of the transmembrane tyrosine kinase can be measured more accurately by detecting the phosphorylation of the substrate.

The substrates for at least 2 types of transmembrane tyrosine kinases may be a mixture of substrates that are highly specific for predetermined transmembrane tyrosine kinases. This mixture contains plural types of substrates different in specificity for transmembrane tyrosine kinases. Grb2 (substrate that is highly specific for HER1), myelin basic protein (MBP), histone H2B (HH2B), phospholipase C-γ, or the like can be used as a substrate that is highly specific for a specific transmembrane tyrosine kinase. A commercial substrate such as a GST EGFR-substrate (Stratagene) can also be used as a substrate that is highly specific for HER1. The GST EGFR-substrate is a fusion protein between GST and a substrate artificially produced so as to be phosphorylated with an enzyme activity of HER1. By using a mixture of different substrates in measurement, the activity values of various transmembrane tyrosine kinases in a sample can be determined.

A universal substrate for plural types of transmembrane tyrosine kinases can be used as the substrates for at least 2 types of transmembrane tyrosine kinases. The universal substrate is a substrate common among plural types of transmembrane tyrosine kinases. That is, the universal substrate is a substrate to be phosphorylated with plural types of transmembrane tyrosine kinases. As the universal substrate, known synthetic peptides are known. Examples include synthetic peptides used as tyrosine kinase substrates in the following literatures: Norio Sasaki et al., 1985, The Journal of Biological Chemistry, Vol. 260, No. 17, 9793-9804; Sergei Braun et al., 1984, The Journal of Biological Chemistry, Vol. 259, No. 4, 2051-2054; and M. Adbel-Ghany et al., 1990, Proceeding of The National Academy of Science, Vol. 87, 7061-7065. A synthetic peptide disclosed in these literatures consists of an amino acid sequence containing a glutamic acid (hereinafter abbreviated as Glu) residue and a tyrosine (hereinafter abbreviated as Tyr) residue. The synthetic peptide is artificially prepared such that Tyr can be phosphorylated by two or more types of tyrosine kinases. A specific amino acid sequence of this synthetic peptide includes:
(1) An amino acid sequence wherein a sequence consisting of 4 Glu residues and 1 Tyr residue is repeated twice or more (referred to hereinafter as amino acid sequence a);
(2) An amino acid sequence wherein a sequence consisting of 1 Glu residue and 1 Tyr residue is repeated twice or more (referred to hereinafter as amino acid sequence b);
(3) An amino acid sequence wherein a sequence consisting of 6 Glu residues, 1 Tyr residue and 3 alanine (hereinafter abbreviated as Ala) residues is repeated twice or more (referred to hereinafter as amino acid sequence c);
(4) An amino acid sequence wherein a sequence consisting of 1 Glu residue, 1 Tyr residue and 1 Ala residue is repeated twice or more (referred to hereinafter as amino acid sequence d); and
(5) An amino acid sequence wherein a sequence consisting of 2 Glu residues, 1 Tyr residue, 6 Ala residues, and 5 lysine (hereinafter abbreviated as Lys) residues is repeated twice or more (referred to hereinafter as amino acid sequence e).
   An acidic amino acid residue is reported to be necessary for phosphorylation of Tyr by a tyrosine kinase (Tony Hunter, 1982, The Journal of Biological Chemistry, Vol. 257, No. 9, 4843-4848). Accordingly, the amino acid sequence a or the amino acid sequence c, which contains many Glu residues that are acidic amino acid residues, is preferable. In measurement of the activity value of transmembrane tyrosine kinases, the universal substrate can be used to determine the comprehensive activity value of plural types of transmembrane tyrosine kinases contained in a sample.

The activity value of transmembrane tyrosine kinases, including the activity value of transmembrane tyrosine kinases of as many types as possible, is preferably measured for more accurate assessment of a risk of cancer relapse. Hence, the universal substrate mentioned above is preferably used.

In an enzyme reaction catalyzed by the transmembrane tyrosine kinase, a phosphorylated group of the phosphate group donor is incorporated into a substrate by the enzyme activity of the transmembrane tyrosine kinase activated through autophosphorylation. The phosphate group donor includes, for example, adenosine triphosphate (ATP), adenosine-5′-O-(3-thiotriphosphate) (ATP-γS), ³²P-labeled adenosine-5′-O-(3-thiotriphosphate) (γ-[³²P]-ATP), adenosine diphosphate (ADP), and adenosine monophosphate (AMP).

The substrate preferably has an affinity tag. The substrate can thereby be recovered by using a solid phase having a binding substance capable of binding to the affinity tag (referred to hereinafter as a solid phase with a binding substance). That is, a complex in which the substance having an affinity tag was bound to the solid phase with a binding substance is recovered. Then, the substrate can be recovered by dissociating the binding between the affinity tag and the solid phase with a binding substance in the recovered complex.

The affinity tag is not particularly limited as long as it is capable of binding to a binding substance and does not interfere with the binding of the substrate to the transmembrane tyrosine kinase or the phosphorylation of the substrate. For example, a polypeptide, a hapten or the like can be used as the affinity tag. Specific examples of the affinity tag that can be used include glutathione-S-transferase (referred to hereinafter as GST), histidine, a maltose-binding protein, FLAG peptide (Sigma), Myc tag, HA tag, Strep tag (IBA GmbH), biotin, avidin, and streptavidin.

The substrate having an affinity tag includes, for example, a fusion protein between a substrate and an affinity tag. The fusion protein may be one having an affinity tag and a substrate thereto. The fusion protein may be a fusion protein produced by a host transformed with a vector harboring a recombinant gene for expressing a fusion protein between the affinity tag and the substrate.

The binding substance is not particularly limited as long as it can be bound to an affinity tag so as to be dissociated from the tag. The binding substance includes, for example, glutathione, nickel, amylose, FLAG antibody (Sigma), Myc antibody, hemagglutinin (HA) antibody, and Strep-Tactin (IBA GmbH).

The solid phase is not particularly limited as long as it is a carrier capable of binding to the binding substance. The material of the solid phase includes, for example, polysaccharides, plastics, glass, and the like. The solid phase is for example in the form of beads, gel, or the like. Specific examples of the solid phase include Sepharose beads, agarose beads, magnetic beads, glass beads, silicone gel, and the like. The beads or gel may be used after being charged into a column.

For example, when GST is selected as the affinity tag, glutathione Sepharose beads (referred to hereinafter as glutathione beads) can be used as the solid phase with a binding substance. That is, a GST-substrate phosphorylated by the enzyme activity of a transmembrane tyrosine kinase is bound to glutathione beads. The beads are recovered, and by adding reduced glutathione thereto, the binding between GST and the glutathione beads can be dissociated. The phosphorylated GST-substrate can thereby be recovered. When the phosphorylated GST-substrate is to be recovered, GST-substrate may be previously bound to glutathione beads and then used in the enzyme reaction. Alternatively, GST-substrate may be bound to glutathione beads after the enzyme reaction.

For example, when histidine is selected as the affinity tag, nickel agarose beads can be used as the solid phase with a binding substance. The binding between histidine and nickel can be dissociated for example with an acid such as glycine-HCl or with imidazole.

For example, when a maltose-bound protein is selected as the affinity tag, amylose magnetic beads can be used as the solid phase with a binding substance. The binding between the maltose-binding protein and amylose can be dissociated for example by adding free amylose.

For example, when FLAG peptide is selected as the affinity tag, FLAG affinity gel (Sigma) can be used as the solid phase with a binding substance. The binding between FLAG peptide and FLAG affinity gel can be dissociated for example with an acid such as glycine-HCl or with 3× FLAG peptide (Sigma).

For example, when Myc tag is selected as the affinity tag, Myc antibody-bound agarose beads can be used as the solid phase with a binding substance. When HA tag is selected as the affinity tag, HA antibody-bound agarose beads can be used as the solid phase with a binding substance. Either the binding between Myc tag and Myc antibody or the binding between HA tag and HA antibody can be dissociated for example by denaturing the protein with an acid or an alkali. At this time, an acid or an alkali that can return the denatured protein to the protein in an original state is preferably selected. Specifically, the acid includes hydrochloride acid, and the alkali includes sodium hydroxide.

For example, when Strep tag is selected as the affinity tag, Strep-Tacin-solid phase gel column (IBA GmbH) can be used as the solid phase with a binding substance. The binding between Strep tag and Strep-Tactin can be dissociated for example with desthiobiotin that reversibly reacts with streptavidin.

After the enzyme reaction of transmembrane tyrosine kinases in the sample with the substrate, the enzyme reaction may be terminated by heating treatment, by cooling treatment, or with EDTA, prior to recovery of the substrate. During the step of recovering the substrate, the enzyme reaction may further proceed. Accordingly, the measurement result may vary from a sample to another. However, this problem can be solved by terminating the enzyme reaction.

For detecting the phosphorylated substrate, a labeling substance is used. Examples of the labeling substance include, but are not limited to, a fluorescent substance, an enzyme and a radioisotope. The fluorescent substance includes, for example, fluorescein, coumarin, eosin, phenanthroline, pyrene, and rhodamine. The enzyme includes, for example, an alkaline phosphatase and a peroxidase. The radioisotope includes, for example, ³²P, ³³P, ¹³¹I, ¹²⁵I, ³H, ¹⁴C and ³⁵S.

For detecting the phosphorylated substance, the labeling substance is bound to the phosphorylated substance. For example, the labeling substance can be bound to the phosphorylated substance by using an antibody having the labeling substance and being capable of binding to the phosphorylated substrate.

Alternatively, an antibody capable of specifically binding to the phosphorylated substrate (referred to hereinafter as a phosphorylated substrate-recognizing antibody), and a labeling substance-containing antibody capable of binding to the phosphorylated substrate-recognizing antibody (referred to hereinafter as second antibody), can be used to bind the labeling substance to the phosphorylated substrate. In this case, the labeling substance can be substantially bound to the phosphorylated substrate via the phosphorylated substrate-recognizing antibody and the second antibody.

Alternatively, a phosphorylated substrate-recognizing antibody, a second antibody having biotin, and avidin having a labeling substance can be used to bind the labeling substance to the phosphorylated substrate. In this case, the labeling substance can be substantially bound to the phosphorylated substrate via the phosphorylated substrate-recognizing antibody, the second antibody, biotin and avidin. The second antibody may have avidin, and biotin may have the labeling substance.

Alternatively, a phosphorylated substance-recognizing antibody having biotin, and avidin having a labeling substance, may be used, or a phosphorylated substrate-recognizing antibody having avidin, and biotin having a labeling substance, may be used.

By detecting a signal emitted by the labeling substance, the phosphorylated substrate can be detected. The activity of the transmembrane tyrosine kinase can thereby be measured.

The phosphorylated substrate-recognizing antibody and the second antibody may be antibodies obtained by contacting an antigen with an animal to promote immunization and purifying blood of the animal, or an antibody, a polyclonal antibody or a monoclonal antibody obtained by genetic recombination. A mixture of at least 2 types of such antibodies may also be used. As used herein, the term “antibody” includes a fragment of an antibody and derivatives thereof. Specific examples include Fab fragment, F(ab′) fragment, F (ab) 2 fragment and sFv fragment and the like (Blazar et al., 1997, J. Immunol., 159: 5821-5833 and Bird et al., 1988, Science, 242: 423-426). The antibody class that can be used herein includes, but is not limited to, IgG and IgM.

The method of detecting the phosphorylated substrate is suitably selected depending on the type of the labeling substance. When the labeling substance is a fluorescent substance, the phosphorylation of the substrate can be detected by western blotting. More specifically, the phosphorylated substrate is separated on a membrane. Then, a phosphorylated substrate-recognizing antibody is added and bound to the phosphorylated substrate on the membrane. Then, a second antibody having a fluorescent substance is bound to the phosphorylated substrate-recognizing antibody bound to the phosphorylated substrate on the membrane. Then, the second antibody having a fluorescent substance, bound to the membrane via the phosphorylated substrate-recognizing antibody, is detected. When the phosphorylated substrate is previously separated by using the affinity tag mentioned above, the phosphorylation of the substrate can be detected by slot blotting in place of western blotting. As the labeling substance, an enzyme can be used in place of the fluorescent substance. When an enzyme is used, an enzyme possessed by a second antibody may be added to the substrate, to cause a luminous reaction, and the luminescence can be detected. A solution containing the phosphorylated substrate is housed in a tube, and a phosphorylated substrate-recognizing antibody having a fluorescent substrate can be added and bound to the phosphorylated substrate, to measure the fluorescence intensity, thereby detecting the phosphorylation of the substrate.

When the labeling substance is an enzyme, the phosphorylation of the substrate can be detected by solid-phase enzyme-linked immunosorbent assay (referred to hereinafter as ELISA). ELISA includes a direct adsorption method and a sandwich method.

In the direct adsorption method, the phosphorylated substrate is adsorbed onto the surface of a solid phase, and a phosphorylated substrate-recognizing antibody having an enzyme is added and bound to the phosphorylated substrate. Then, the enzyme possessed by the phosphorylated substrate-recognizing antibody is added to the substrate to cause a luminous reaction, and the luminescence is detected.

In the sandwich method, a phosphorylated substrate-recognizing antibody is bound to a solid phase (referred to hereinafter as a solid-phase antibody) and added to the phosphorylated substrate to allow it to react with the solid-phase antibody. Then, a phosphorylated substrate-recognizing antibody having an enzyme (referred to hereinafter as a labeled antibody) is added and bound to the phosphorylated substrate. The enzyme possessed by the labeled antibody is added to the substrate to cause a luminous reaction, and the luminescence is detected.

For example, when the enzyme is an alkaline phosphatase, a mixed solution of nitrotetrazolium blue chloride (NBT) and 5-bromo-4-chloro-3-indoxyl phosphate (BCIP) can be used as the substrate to cause coloring. When the enzyme is a peroxidase, diaminobenzidine (DAB) can be used as the substrate to cause coloring.

When the sandwich method is used, the solid-phase antibody and the labeled antibody are bound preferably to different sites of the phosphorylated substrate. That is, it is preferable that the phosphorylated substrate have a plurality of antibody-binding sites, or 2 types of antibodies to be used recognize different antigenic determinants of the phosphorylated substrate.

When the labeling substance is a radioisotope, the phosphorylation of the substrate can be detected by radioimmunoassay (referred to hereinafter as RIA). Specifically, a phosphorylated substrate-recognizing antibody having a radioisotope is bound to the phosphorylated substrate, and its radiation can be measured with a scintillation counter or the like to detect the phosphorylation of the substrate.

In this manner, the comprehensive activity value of various transmembrane tyrosine kinases in a sample is measured in the method in the present embodiment. Then, the obtained comprehensive activity value reflects the activity of various types of transmembrane tyrosine kinases occurring in cells. It follows that when the comprehensive activity value of transmembrane tyrosine kinases is measured by using cells in a tumor tissue collected from a cancer patient, a risk of cancer relapse can be accurately evaluated on the basis of the obtained comprehensive activity value of the membrane transmembrane tyrosine kinases.

A risk of cancer relapse can be evaluated on the basis of the comprehensive activity value of transmembrane tyrosine kinases in a sample determined in the method described above. Specifically, the obtained comprehensive activity value of transmembrane tyrosine kinases is compared with a predetermined threshold value, and when the activity value is lower than the threshold value, the risk of cancer relapse can be determined to be low.

The threshold value is suitably determined depending on the type and the like of a tumor cell to be measured. For example, the threshold value can be established based on the comprehensive activity value of transmembrane tyrosine kinases obtained from a plurality of cancer patients who are known for the presence or absence of a relapse. Specifically, on the basis of both information on the comprehensive activity value of transmembrane tyrosine kinases in cells of malignant tumors collected from a plurality of cancer patients and information on the presence or absence of relapses of the cancer patients, the comprehensive threshold value of transmembrane tyrosine kinases that can distinguish the cancer patients into groups different in relapse risk (for example, a group of high relapse risk and a group of low relapse risk) can be selected and used as the threshold value. For example, when the subject to be measured is a breast cancer-derived tumor cell, the threshold value can be established by the following method. On the basis of both information on the comprehensive activity value of transmembrane tyrosine kinases of breast cancer cells collected from a plurality of breast cancer patients and information on the presence or absence of relapses of the breast cancer patients in 5 years after extirpative surgery, the comprehensive threshold value of transmembrane tyrosine kinases that can distinguish the cancer patient into groups different in relapse risk (for example, a group of high relapse risk and a group of low relapse risk) may be selected.

By establishing a plurality of threshold values, the risk of cancer relapse can be evaluated to classify the cancer patients into groups such as a group of high relapse risk (high-risk group), a group of intermediate relapse risk (intermediate-risk group) and a group of low relapse risk (low-risk group).

As described above, transmembrane tyrosine kinases are involved in malignant transformation of cells. The comprehensive activity value of transmembrane tyrosine kinases is an activity value of various types of transmembrane tyrosine kinases occurring in cell membranes. From the foregoing, the comprehensive activity value of transmembrane tyrosine kinases can be used in accurate assessment of the malignant transformation of tumor cells.

Accordingly, the risk of relapse can be accurately determined by using the determining method in the first embodiment.

The determining method in the second embodiment of the present invention comprises determining a risk of cancer relapse on the basis of the comprehensive activity value of transmembrane tyrosine kinases in a tumor cell and a parameter of a cyclin-dependent kinase (CDK) obtained from the expression level and activity value of CDK in the tumor cell.

The determination is performed by comparing the comprehensive activity value of transmembrane tyrosine kinases with a first threshold value and comparing the CDK parameter with a second threshold value.

The comparison of the comprehensive activity value of transmembrane tyrosine kinases with the first threshold value can be performed in the same manner as in the first embodiment. In this embodiment, the “threshold value” shown in the first embodiment is expressed as the “first threshold value”.

In this embodiment, the CDK parameter is further compared with a second threshold value.

This CDK parameter may be a parameter of a cyclin-dependent kinase (CDK) in a tumor cell, obtained based on the expression level and activity value of CDK. The CDK parameter includes, for example, the ratio (CDK specific activity) of the activity value to expression level of CDK in a tumor cell. The CDK parameter further includes the ratio (CDK specific activity ratio) of a first cyclin-dependent kinase (first CDK) specific activity to a second cyclin-dependent kinase (second CDK) specific activity, obtained based on the ratio (first CDK specific activity) of the activity value to expression level of the first CDK in a tumor cell and the ratio (second CDK specific activity) of the activity value to expression level of the second CDK in the tumor cell. The CDK parameter is more preferably a CDK specific activity ratio.

The CDK is known as a parameter that accurately reflects the state of a malignant tumor in a cancer patient. The CDK is a collective term of enzymes activated through binding of a protein called the cyclin and functions in a particular stage of a cell cycle depending on the type thereof. The type of the CDK includes CDK1, CDK2, CDK3, CDK4, CDK5, CDK6, CDK7 and the like. In the present embodiment, at least two types of CDKs (first CDK and second CDK) are used, and a combination of CDK1 and CDK2 is particularly preferably used. That is, it is preferable that the first CDK be CDK1 while the second CDK be CDK2, or the first CDK be CDK2 while the second CDK be CDK1.

The CDK parameter is preferably a combination of CDK1 specific activity and CDK2 specific activity or the ratio of CDK2 specific activity to CDK1 specific activity (CDK specific activity ratio), particularly preferably the ratio of CDK2 specific activity to CDK1 specific activity (CDK specific activity ratio).

The first CDK specific activity is the ratio of the activity value to expression level of a first cyclin-dependent kinase (first CDK) in a tumor cell. More specifically, the first CDK specific activity is a value (activity value/expression level=specific activity) obtained by dividing the activity value of the first CDK by the expression level thereof. The second CDK specific activity is the ratio of the activity value to expression level of a second cyclin-dependent kinase (second CDK) in a tumor cell. More specifically, the second CDK specific activity is a value (activity value/expression level=specific activity) obtained by dividing the activity value of the second CDK by the expression level thereof.

The CDK specific activity ratio is a ratio between the first CDK specific activity and the second CDK specific activity. The ratio between the first CDK specific activity and the second CDK specific activity includes both a value obtained by dividing the first CDK specific activity by the second CDK specific activity and a value obtained by dividing the second CDK specific activity by the first CDK specific activity.

The CDK specific activity ratio is obtained by the following steps: a step of measuring the activity values of the first and second CDKs from a tumor cell, a step of measuring the expression levels of the first and second CDKs from the tumor cell, a step of calculating the activity value/expression level ratio of the first CDK (first CDK specific activity) and the activity value/expression level ratio of the second CDK (second CDK specific activity), and a step of calculating the ratio (CDK specific activity ratio) between the first CDK specific activity and the second CDK specific activity, which are obtained by the specific activity ratio calculating step.

The second threshold value is suitably determined depending on e.g. a tumor cell to be measured. For example, the second threshold value can be established based on a CDK parameter obtained from a plurality of cancer patients who are known for the presence or absence of a relapse. Specifically, on the basis of both information on the CDK parameter of tumor cells collected from a plurality of cancer patients and information on the presence or absence of relapses of the cancer patients, the CDK parameter value that can distinguish the cancer patients into groups different in relapse risk (for example, a group of high relapse risk and a group of low relapse risk) can be selected and used as the second threshold value. For example, when the subject to be measured is a breast cancer-derived tumor cell, the second threshold value can be established by the following method. On the basis of both information on the CDK parameter of breast cancer cells collected from a plurality of breast cancer patients and information on the presence or absence of relapses of the breast cancer patients in 5 years after extirpative surgery, the CDK parameter value that can distinguish the breast cancer patients into a group of high relapse risk (high-risk group) and a group of low relapse risk (low-risk group) may be selected.

By combination with the first threshold value, the CDK parameter value that can distinguish the cancer patients into groups different in relapse risk may be selected and used as the second threshold value. For example, when the subject to be measured is a breast cancer-derived tumor cell, the threshold value can be established in the following method. The CDK parameter value that can further distinguish the high-risk group and/or the low-risk group distinguished with the first threshold value, into a group of high relapse risk (high-risk group) and a group of low relapse risk (low-risk group) may be used as the second threshold value.

By establishing a plurality of second threshold values, the risk of cancer relapse can be evaluated so that the cancer patients are classified into multiple groups such as a group of high relapse risk (high-risk group), a group of intermediate relapse risk (intermediate-risk group) and a group of low relapse risk (low-risk group).

A measurement sample used in the measurement of the activity value and expression level of CDK is prepared by using the cell in the tumor tissue as used in measurement of the comprehensive activity value of transmembrane tyrosine kinases. A method for preparing the sample is not particularly limited as far as the activity value and expression level of the CDK can be measured. For example, a sample in which the cell is solubilized (hereinafter referred to as cell lysate) can be used as the measurement sample. The cell lysate is a sample prepared by adding a buffer to a tumor tissue collected from a cancer tissue and physically and/or chemically crushing tumor cancers in the tumor tissue in the buffer. When the activity value or expression level of the CDK is measured, a supernatant obtained by centrifuging the cell lysate is used preferably as the measurement sample. The buffer used herein may appropriately contain a surfactant, a protease inhibitor, and the like.

The method of measuring the activity value and expression level of the CDK is not particularly limited, and a conventionally known method can be used. For example, the methods described in U.S. Patent Application Publication Nos. 2007-231837 and 2007-141651 can be used.

The determination of relapse risk is performed by comparing the comprehensive activity value of transmembrane tyrosine kinases with the first threshold value and comparing the CDK parameter with the second threshold value. For example, the risk of cancer relapse can be determined to be low when the comprehensive activity value of transmembrane tyrosine kinases is lower than the first threshold value and simultaneously when the CDK parameter is lower than the second threshold value.

As described above, the comprehensive activity value of transmembrane tyrosine kinases can be used in accurate assessment of the malignant transformation of tumor cells.

Further, the CDK parameter can be used in accurate assessment of the proliferative ability of tumor cells because the CDK is really involved in proliferation of cells. The proliferative ability of cells is one major factor for determining a risk of cancer relapse.

Accordingly, the determining method in the second embodiment can be used to determine a risk of relapse more accurately by using both the comprehensive activity value of transmembrane tyrosine kinases and the CDK parameter.

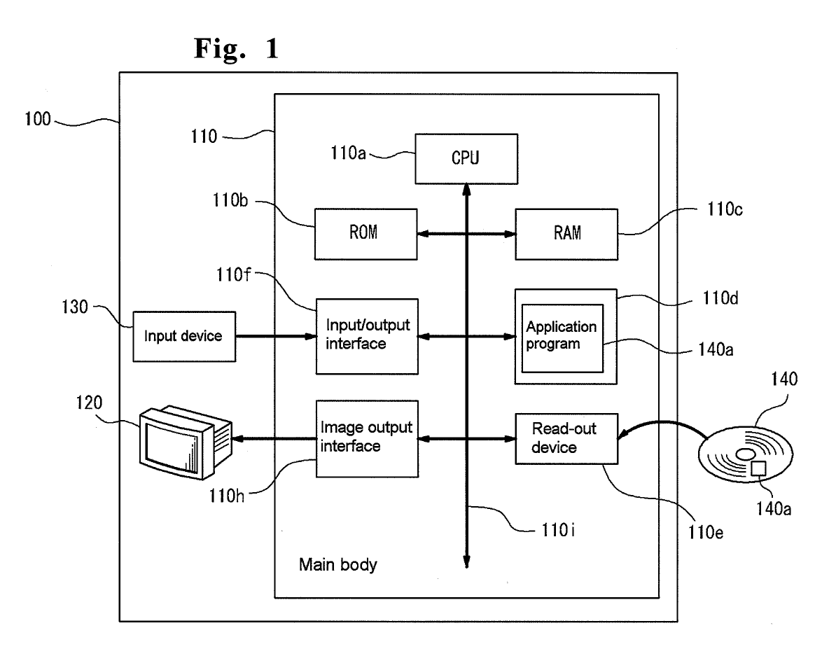

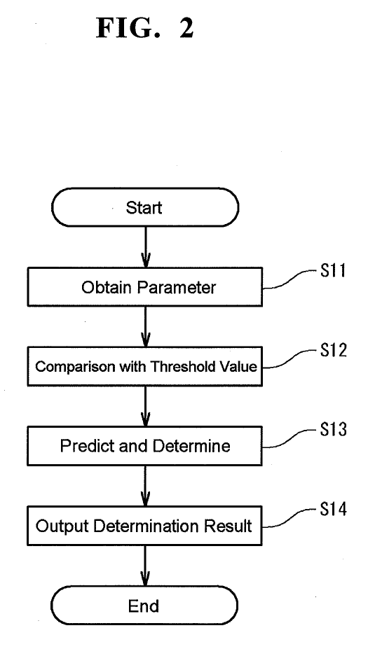

The method in the present embodiment is performed preferably in a computer. Hereinafter, a computer system (FIG. 1) and a judgment flow (FIG. 2) for performing the second determining method will be described. In the determining method in the second embodiment described herein, the ratio (CDK specific activity ratio) between CDK1 specific activity and CDK2 specific activity is used as the CDK parameter.

The system 100 shown in FIG. 1 includes a computer body 110, an input device 130 for entering necessary data to the computer body 110, and a display 120 for displaying input-output data and the like and can further optionally include an external recording medium 140. A program 140a in this embodiment may be recorded on the external recording medium 140. Alternatively, the program 140a may be stored in memories 110b to 110d installed in the computer body 110. CPU 110a, memories 110b to 110d, an input-output interface 110f, an image output interface 110h, and a read-out device 110e are connected to one another via bus 110i in the computer body 110 such that data can be transmitted and received.

FIG. 2 is a flowchart showing the operation of a program for executing the determination of a risk of relapse according to the first determining method. This program is stored in a memory (hard disk) 110d.

When the comprehensive activity value of transmembrane tyrosine kinases in a sample (referred to hereinafter as the first parameter) and the ratio of CDK2 specific activity to CDK1 specific activity (CDK2 specific activity/CDK1 specific activity: CDK specific activity ratio) (referred to hereinafter as the second parameter) are input with the input unit 130, CPU 110a obtains these parameters through the I/O interface 110f and allows RAM 110c to memorize these parameters (Step S11).

The CPU 110a previously reads the first threshold value corresponding to the first parameter stored in the memory (hard disk) 110d as the program data, and the second threshold value corresponding to the second parameter, respectively. Then, the CPU 110a executes comparison between the first threshold value and the first parameter data and comparison between the second threshold value and the second parameter data (Step S12).

Next, the CPU 110a predicts and determines a risk of relapse based on the comparison results (Step S13). The CPU 110a determines the risk of relapse is “Low” when the first parameter is below the first threshold value and the second parameter is below the second threshold value.

Then, the CPU 110a stores the determination result in the RAM 110c and outputs the determination result to the display 120 via the image output interface 110h (Step S14).

In the present embodiment, the respective parameter data, activity value and the like are inputted via the input unit 130; however, the input method is not limited thereto. For example, the data are not necessarily inputted by an operator, and the respective parameter data, activity value, and the like may be automatically obtained from a measuring device via the input/output interface 110f. Further, the value of the CDK2 specific activity/CDK1 specific activity is inputted as the CDK specific activity ratio. As an alternative method, the expression levels and the activity values of the CDK1 and CDK2 are inputted, and the value of the CDK2 specific activity/CDK1 specific activity is calculated from these values by the CPU 110a from these values, so that the obtained value can be used in comparison with the threshold values. The output of the display 120 is not limited to the determination result; for example, the determination result may be outputted together with the result of the first and second parameters obtained by comparison with the respective threshold values.

### EXAMPLE

In the following examples, a universal substrate for plural types of transmembrane tyrosine kinases was used. First, a method of preparing the substrate is described.

### Preparation of the Substrate for Tyrosine Kinases

A fusion protein between GST and a peptide consisting of an amino acid sequence (SEQ ID NO: 1) wherein a sequence composed of 4 glutamic acid residues and 1 tyrosine residue was repeated 5 times (referred to hereinafter as a poly(Glu, Tyr) peptide) was prepared. Then, this fusion protein was used as a substrate that can be phosphorylated with any types of transmembrane tyrosine kinases. Hereinafter, this fusion protein is referred to as GST-poly(Glu, Tyr) substrate.

The GST-poly(Glu, Tyr) substrate was prepared in the following manner. PCR was conducted using KOD plus DNA polymerase (Toyobo) as well as a sense primer (SEQ ID NO: 3) and an antisense primer (SEQ ID NO: 4) designed on the basis of a base sequence of a DNA (SEQ ID NO: 2) encoding an amino acid sequence (SEQ ID NO: 1) of the poly(Glu, Tyr) peptide. An amplification product (referred to herein after as poly(Glu, Tyr) DNA) obtained by PCR, and a GST fusion protein expression plasmid vector pGEX-4T-3 (GE Healthcare Bioscience), were treated with restriction enzymes (BamH1 and EcoR1), followed by integrating the poly(Glu, Tyr) DNA into pPEX-4T-3, to prepare a recombinant plasmid. This recombinant plasmid was transformed into Escherichia coli JM109, and this E. coli was cultured in a liquid medium (LB medium) until the absorbance (600 nm) of the culture reached 0.6. The cultured E. coli was supplemented with 1 mM of IPTG (concentration in the culture) and cultured for 4 hours to induce expression. Then, the E. coli was lysed, and a GST-poly(Glu, Tyr) substrate was recovered with glutathione Sepharose 4B (GE Healthcare Bioscience). The amino acid sequence of the GST-poly(Glu, Tyr) substrate is set forth in SEQ ID NO: 5.

Then, the prepared GST-poly(Glu, Tyr) substrate was confirmed to be phosphorylated with various transmembrane tyrosine kinases.

### Reference Example

### Detection of the Phosphorylation of the GST-poly(Glu, Tyr) Substrate

The GST-poly(Glu, Tyr) substrate was phosphorylated by an intracellular domain (ICD) of a commercial transmembrane tyrosine kinase, and the phosphorylated GST-poly(Glu, Tyr) substrate was detected by western blotting. The transmembrane tyrosine kinase is composed of an extracellular domain, a transmembrane domain and an intracellular domain, among which the intracellular domain contains an active site of the tyrosine kinase.

### (Preparation of a Reaction Sample)

50 μl of buffer 1 (containing 20 mM HEPES, pH 7.4, 10 mM MnCl₂, 1% NP-40, 1 mM DTT, 0.2% protease inhibitor (referred to hereinafter as PI), 10% glycerol, 200 μM Na₃VO₄ and 50 mM NaF) was mixed with 0.5 pmol of an ICD of a commercial transmembrane tyrosine kinase and used as a reaction sample in the following enzyme reaction. The ICD used herein was an ICD of each of PDGF receptor β kinase (referred to hereinafter as PDGFR-β), VEGF receptor 1 kinase (referred to hereinafter as VEGFR1), VEGF receptor 2 kinase (referred to hereinafter as VEGFR2), EGF receptor 1 kinase (referred to hereinafter as HER1), ErbB2 kinase (referred to hereinafter as HER2), ErbB4 kinase (referred to hereinafter as HER4) and IGF-1 receptor kinase (referred to hereinafter as IGF1R) (all of which were from Cell Signaling Technology). A reaction sample i was a mixture of buffer 1 and PDGFR-β, a reaction sample ii was a mixture of buffer 1 and VEGFR1, a reaction sample iii was buffer 1 and VEGFR2, a reaction sample iv was a mixture of buffer 1 and HER1, a reaction sample v was a mixture of buffer 1 and HER2, a reaction sample vi was a mixture of buffer 1 and HER4, and a reaction sample vii was a mixture of buffer 1 and IGF1R.

### (Enzyme Reaction)

25 μl of the reaction sample i was mixed with 25 μl of substrate solution 1 containing the GST-poly(Glu, Tyr) substrate (containing 20 mM HEPES, pH 7.4, 10 mM MnCl₂, 1 mM DTT, 1% NP-40, 0.2% PI, 10% glycerol, 200 μM Na₃VO₄, 50 mM NaF, 40 μM ATP, and 5 μg GST-poly(Glu, Tyr) substrate) and then incubated at 25° C. for 60 minutes. To this reaction solution was added 25 μl of an SDS sample buffer, pH 6.8 (containing 200 mM Tris, 40% glycerol, 8% SDS, and 10% 2-mercaptoethanol), and the mixture was boiled at 100° C. for 5 minutes to terminate the enzyme reaction. The solution thus prepared was used as SDS sample i (+). In the same manner, SDS samples ii (+) to vii (+) were prepared from the reaction samples ii to vii.

Separately, 25 μl of the reaction sample i was mixed with ATP-free substrate solution 2 (containing 20 mM HEPES, pH 7.4, 10 mM MnCl₂, 1 mM DTT, 1% NP-40, 0.2% PI, 10 glycerol, 200 μM Na₃VO₄, 50 mM NaF, and 5 μg GST-poly(Glu, Tyr) substrate) and then incubated at 25° C. for 60 minutes. 25 μl of the SDS sample buffer was added to this reaction solution, and the mixture was boiled at 100° C. for 5 minutes to terminate the enzyme reaction. The solution thus prepared was used as SDS sample i (−). In the same manner, SDS samples ii (−) to vii (−) were prepared from the reaction samples ii to vii. The substrate solution 2 has the same composition as in the substrate solution 1 except that ATP is not contained in the substrate solution 2. Then, the SDS samples ii (−) to vii (−) were used as negative controls of the SDS samples ii (+) to vii (+).

### (Detection of the Phosphorylated GST-Poly(Glu, Tyr) Substrate)

Each SDS sample was put onto a polyacrylamide gel (PAGE Mini “Daiichi” 4/20 (13 W), Daiichi Pure Chemicals Co., Ltd.) on each well and then electrophoresed at 25 mA for 70 minutes in an electrophoresis bath (cassette electrophoresis bath “Daiichi” DPE-1020 (mini double-barreled), Daiichi Pure Chemicals Co., Ltd.). The protein separated by electrophoresis was transferred from the polyacrylamide gel onto a polyvinylidene fluoride (PVDF) membrane (Immobilon-FL, 0.45 μm pore size, Millipore Corporation) at a voltage of 100 V for 1 hour by means of the Mini Trans-Blot cell transfer system (Bio-Rad Laboratories, Inc.). This PVDF membrane was blocked with 4% Block Ace (Dainippon Sumitomo Pharma Co., Ltd.) solution. The blocked PVDF membrane was shaken for 60 minutes in 2 ml of a first antibody solution (containing 0.4% Block Ace and 0.5 μg/ml Anti-Phosphotyrosine Clone 4G10 (Upstate Biotech, Inc.)) and then washed 3 times with TBS-T (containing 25 mM Tris, 150 mM NaCl, and 0.1% Tween-20). Then, this PVDF membrane was shaken for 60 minutes in 2 ml of a second antibody solution (containing 0.4% Block Ace and 2.7 μg/ml anti-mouse immunoglobulin rabbit polyclonal antibody labeled with FITC (DAKO Ltd.)) and then washed 3 times with TBS-T. This PVDF membrane was dried and analyzed with an image analyzer (Pharos FX system (Bio-Rad Laboratories, Inc.), to detect fluorescence. In this manner, the phosphorylated GST-poly(Glu, Tyr) substrate contained in each of the SDS samples i (+) to vii (+) and SDS samples i (−) to vii (−) was detected.

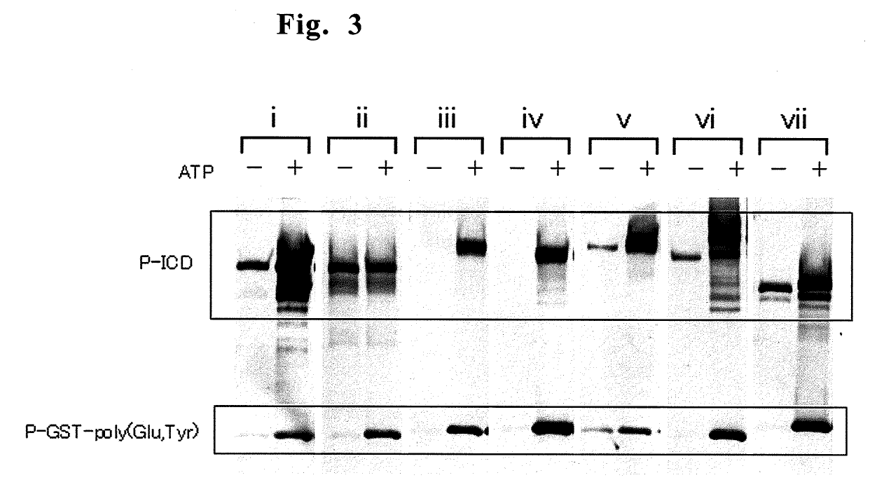

FIG. 3 is a fluorescence photograph showing the result of western blotting. In this photograph, i shows a result with PDGFR-β as a transmembrane tyrosine kinase; ii, a result with VEGFR1; iii, a result with VEGFR2; iv, a result with HER1;, v, a result with HER2; vi, a result with HER4; and vii, a result with IGF1R. In each of the photographs i to vii, “−” shows a result from SDS sample prepared with the ATP-free substrate solution 2, while “+” shows a result from SDS sample prepared with the ATP-containing substrate solution 1. P-ICD shows the position at which the autophosphorylated transmembrane tyrosine kinase appears, and P-GST-poly(Glu, Tyr) shows the position at which the phosphorylated GST-poly(Glu, Tyr) substrate appears.

In every “+” in i to vii in FIG. 3, a single band was observed at the position at which the phosphorylated GST-poly(Glu, Tyr) substrate appears. It was thereby found that the GST-poly(Glu, Tyr) substrate prepared in (1) above can be phosphorylated with many types of transmembrane tyrosine kinases.

In “−” in ii, iii, iv, vi and vii in FIG. 3, no band was observed at the position at which the phosphorylated GST-poly(Glu, Tyr) substrate appears, probably because the reaction solution in the enzyme reaction did not contain ATP so that the GST-poly(Glu, Tyr) substrate was not phosphorylated. In “−” in i and v in FIG. 3, on the other hand, a very pale band was observed at the position at which the phosphorylated GST-poly(Glu, Tyr) substrate appears; this is possibly due to unspecific binding of the antibody used for detection or because the product used in measurement was contaminated with a very small amount of ATP.

### Example 1

In this example, reaction samples containing transmembrane tyrosine kinases from tumor cells collected from cancer patients were prepared, and the GST-poly(Glu, Tyr) substrate was used to measure the comprehensive activity value of transmembrane tyrosine kinases in the measurement samples, and on the basis of this activity value, a risk of cancer relapse was determined.

### (1) Preparation of Reaction Samples

From 31 tumor tissue samples (about 8 to 125 mm³) excised from 31 breast cancer patients (patients 1 to 31), reaction samples 1 to 31 were prepared in the following manner. Pathologist's assessment results of the 31 samples (TMN classification, lymph node metastasis condition, cancer tissue size, presence or absence of relapse within 5 years after surgery, relapse site) are shown in Table 1. The patients 1 to 31 have not only an early stage breast cancer (Stage I or IIA) but also an advanced cancer (Stage IIB or IIIA).

TABLE 1

| Patient No. | TMN Classification | LN | T | Relapse | Relapse Site |
|---|---|---|---|---|---|
| 1 | IIB | b | b | No | |
| 2 | IIA | b | a | No | |
| 3 | IIA | a | c | No | |
| 4 | IIB | a | a | No | |
| 5 | IIA | a | a | Yes | Lymph node |
| 6 | IIA | a | b | No | |
| 7 | IIB | a | b | No | |
| 8 | IIIA | c | b | No | |
| 9 | IIA | a | b | Yes | Lung |
| 10 | IIIA | c | b | Yes | Bone |
| 11 | IIIA | c | c | No | |
| 12 | IIA | a | b | No | |
| 13 | IIA | a | a | No | |
| 14 | IIA | a | b | No | |
| 15 | IIB | a | a | No | |
| 16 | IIB | b | b | No | |
| 17 | IIB | b | a | Yes | Bone |
| 18 | IIB | b | b | No | |
| 19 | I | a | a | No | |
| 20 | IIIA | c | a | Yes | Bone |
| 21 | IIA | a | b | Yes | Lung |
| 22 | IIA | a | b | Yes | Lung |
| 23 | IIA | a | b | Yes | Thoracic wall |
| 24 | IIA | a | b | No | |
| 25 | IIA | b | a | Yes | Lymph node |
| 26 | IIB | b | b | No | |
| 27 | IIIA | c | b | Yes | Bone |
| 28 | IIA | a | b | No | |
| 29 | IIA | a | b | No | |
| 30 | IIIA | c | b | No | |
| 31 | IIA | a | b | No | |

In Table 1, “LN” indicates a condition of lymph node metastasis after surgery, wherein “a” means that no metastasis was observed on regional lymph nodes, “b” means that metastasis was observed on one to three regional lymph nodes, and “c” means that metastasis was observed on four or more regional lymph nodes. “T” indicates the primary tumor size at the time of surgery, wherein “a” means a tumor size of 2 cm or smaller, “b” means a tumor size from 2 cm to 5 cm, and “c” means a tumor size of 5 cm or larger.

A method of preparing the reaction samples 1 to 31 is as follows:

Each tumor tissue was mixed with 1 ml of cell treatment solution (containing 20 mM HEPES, pH 7.4, 0.2% PI, 10% glycerol, 200 μM Na₃VO₄, and 50 mM NaF), and cell membranes were crushed by pressurization with a pestle, to prepare a cell solution. The resulting cell solution was centrifuged, and its supernatant was discharged, while precipitates were recovered. The recovered precipitates were mixed with a cell membrane lysing solution (containing 20 mM HEPES, pH 7.4, 1% NP-40, 0.2% protease inhibitor cocktail (Sigma), 10% glycerol, 200 μM Na₃VO₄, and 50 mM NaF), and the cell membranes were lysed by pressurization with a pestle, followed by centrifugation to recover a supernatant. This supernatant was used as a reaction sample.

### (2) Kinase Reaction

50 μl of reaction solution (containing 20 mM HEPES, pH 7.4, 200 μM Na₃VO₄, 50 mM NaF, 1 mM DDT, 10 mM MnCl₂, 0.2% PI, 10% glycerol, and 200 μM ATP) was introduced into each tube, and 10 μg of one of the reaction samples 1 to 31 prepared in (1) above and 5 μg of the GST-poly(Glu, Tyr) substrate prepared in Reference Example (1) were added to each tube and incubated at 25° C. for 30 minutes. After incubation, 50 μl of 10 mM EDTA was added to terminate the reaction. The solutions thus obtained were used as measurement samples 1 to 31.

### (3) SDS-PAGE and Western Blotting

33 μl of 4× SDS-PAGE sample buffer was added to each of the measurement samples 1 to 31 and heated at 100° C. for 5 minutes. Then, the heated measurement samples 1 to 31 were injected in a volume of 13 μl per well into different wells of a pre-cast gel (PAGE Mini Daiichi 4/20) set in an electrophoresis chamber (Cat. No. 303111, Bio-Rad) and then electrophoresed. After electrophoresis, the wells were detached from a glass plate, and the protein on the gel was transferred onto an Immobilon-FL membrane (Cat. No. S1EJ084E03) manufactured by Millipore Corporation. The membrane onto which the protein had been transferred was dipped in Block Ace (Cat. No. UK-B80, Dainippon Sumitomo Pharma Co., Ltd.), and blocked for 1 hour. Thereafter, the membrane was washed 3 times each for 5 minutes with 1× TBS-T (20 mM Tris-HCl, pH 7.4, 150 mM NaCl, 0.1% Tween20). The membrane was shaken for 1 hour at room temperature in a first antibody (anti-phosphotyrosine clone 4G10 (Cat. No. 05-321) Upstate Biotech, Inc.) solution diluted 2000-fold with a 1/10 dilution of Block Ace. Then, the membrane was washed 3 times each for 5 minutes with 1× TBS-T. Then, the membrane was shaken for 1 hour at room temperature in a second antibody (polyclonal rabbit anti-mouse immunogloblins/FITC (Cat. No. F0261, DAKO Ltd.)) solution diluted 1000-fold with a 1/10 dilution of Block Ace, and then washed 3 times each for 5 minutes with 1× TBS-T. The membrane was dried, and then its signal was detected with an imager (MOLECULAR IMAGER FX, manufactured by Bio-Rad Laboratories, Inc.), and the density of a band was numerically expressed as the activity value.

### (4) Establishment of Threshold Value

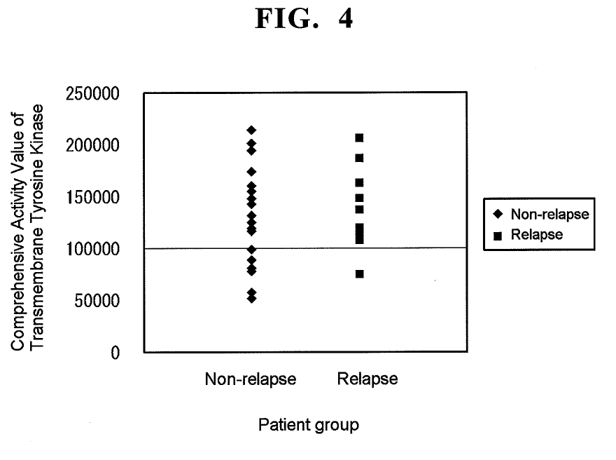

The 31 breast cancer patients were divided into a relapse group and a non-relapse group, depending on whether they had a relapse in 5 years after surgery, and a graph was prepared by plotting the measured comprehensive activity values of transmembrane tyrosine kinases from the 31breast cancer patients on the ordinate against the patient group (abscissa). The graph is shown in FIG. 4. Then, the activity value that can separate the relapse group and the non-relapse group into a high-value group and a low-value group respectively was determined as a threshold value. The threshold value was 100000, and it was determined that the risk of relapse is low (Relapse Risk “Low”) when the comprehensive activity of transmembrane tyrosine kinases is lower than the threshold value (100000).

### (5) Determination of a Risk of Relapse

From the results shown in Table 1 and FIG. 4, 10 patients out of the 31 breast cancer patients had a relapse in 5 years after surgery (relapse rate: 32%). On the other hand, when the risk of relapse was determined based on the comprehensive activity value of transmembrane tyrosine kinases, 7 patients out of the 31 breast cancer patients were judged to be “low” in the risk of relapse from the results shown in FIG. 4, and out of the 7 patients, 1 patient had a relapse in 5 years after surgery (relapse rate: 14%).

From the foregoing, a group with a low risk of relapse can be classified by comparing the comprehensive activity value of transmembrane tyrosine kinases with the threshold value. Whether a relapse occurs or not can also be predicted. This prediction result can serve as an indicator not only for determining an early cancer but also for deciding on courses of treatment of patients with an advanced cancer.

### Example 2

In this example, tumor cancers collected from the same cancer patients as in Example 1 were used and measured for the comprehensive activity value of transmembrane tyrosine kinases and for CDK specific activity ratio, and the risk of cancer relapse was determined on the basis of the comprehensive activity value and the CDK specific activity ratio.

The comprehensive activity value of transmembrane tyrosine kinases was the same value as measured in Example 1.

### (1) Preparation of CDK Measurement Samples

The 31 tumor tissues used in Example 1 and buffer A (containing 0.1 w/v % Nonidet P-40 (Calbiochem Corporation), 50 mM Tris-HCl, pH 7.4, 5 mM EDTA, 50 mM NaF, 1 mM Na₃VO₄ and 100 μl/ml protease inhibitor cocktail (Sigma)) were introduced into tubes in an amount of about 150 mg/ml of tumor tissue in the buffer.

The tumor tissue was homogenized in the buffer A with an electric homogenizer to crush tumor cells to prepare a cell lysate. Then, the cell lysate was centrifuged at 15000 rpm at 4° C. for 5 minutes, and the supernatant was used as a CDK measurement sample.

### (2) Measurement of Expression Levels of CDK1 and CDK2

50 μl each of the CDK measurement samples was housed in each well of a blotter set in a PVDF membrane (Millipore Corporation). Then, the CDK measurement sample was suctioned from a bottom surface of the well, which is a rear surface of the membrane, at a negative pressure of about 250 mmHg for about 30 seconds so that protein in the CDK measurement sample was adsorbed onto the membrane.

100 μl of a washing solution B (containing 25 mM Tris-HCl, pH 7.4 and 150 mM NaCl) was housed in each well and suctioned at the negative pressure of 500 mmHg for 15 seconds so that the membrane was washed.

After the washing process, 40 μl of a blocking reagent B (containing 4% BSA, 25 mM Tris-HCl, pH 7.4 and 150 mM NaCl) was housed in each well and left in a stationary state for 15 minutes, and suctioned at the negative pressure of 500 mmHg for 15 seconds so that the membrane was blocked.

After the blocking process, 40 μl of a rabbit anti-CDK1 antibody (first antibody: Santa Cruz Biotechnology Inc.) which is specifically bound with the CDK1 was housed in each well and left in the stationary state at room temperature for about 30 minutes so that the CDK1 in the membrane and the first antibody were reacted with each other. Then, the suction was applied thereto from the bottom surface of the well at the negative pressure of 500 mmHg for about 15 seconds.

100 μl of the washing solution B was housed in each well and suctioned at the negative pressure of 500 mmHg for 15 seconds so that the membrane was washed.

40 μl of a biotinylated anti-rabbit CDK1 antibody (second antibody: Santa Cruz Biotechnology Inc.) solution was housed in each well and left in the stationary state at room temperature for about 30 minutes so that the first antibody was reacted with second antibody in the membrane. Thereafter, the suction was applied thereto from the bottom surface of the well at the negative pressure of 500 mmHg for about 15 seconds.

100 μl of the washing solution B was housed in each well and suctioned at the negative pressure of 500 mmHg for 15 seconds so that the membrane was washed.

50 μl of a label solution containing FITC-labeled streptavidin was housed in each well and left in the stationary state at room temperature for about 30 minutes so that the second antibody in the membrane was labeled with FITC. Thereafter, the suction was applied thereto from the bottom surface of the well at the negative pressure of 500 mmHg for about 15 seconds.

50 μl of the washing solution B was housed in each well and suctioned at the negative pressure of 500 mmHg for 15 seconds, which was repeated five times so that the membrane was washed.

The membrane was removed from the blotter, washed with 20% methanol for about 5 minutes and dried at room temperature for about 20 minutes. Thereafter, fluorescence intensity of the protein adsorbed onto the membrane was analyzed and measured by Molecular Imager FX (Bio-Rad Laboratories Inc.) which was a fluorescence image analyzer. The measurement value was calculated based on a calibration curve.

The calibration curve was prepared as follows: 50 μl of a solution obtained by dissolving CDK1 at five different concentrations in a washing solution B containing 0.005% Nonidet P-40 and 50 μg/ml of BSA was injected into a well treated in the same manner as described above and then labeled with FITC in the same experimental procedure as described above, and fluorescence intensity thereof was measured, to prepare a calibration curve showing the relationship between the fluorescence intensity and the expression level of the CDK1.

The expression level of the CDK2 was measured in an experimental procedure similar to the measurement of the expression level of the CDK1 described earlier except that a rabbit anti-CDK2 antibody was used as the first antibody in place of the rabbit anti-CDK1 antibody.

### (3) Measurement of Activities of CDK1 and CDK2

500 μl of buffer A was introduced into a 1.5-ml of Eppendorf tube, and the CDK measurement sample was added thereto. The CDK measurement sample was prepared and added to the Eppendorf tube such that the total amount of proteins in the mixed solution in the tube was 100 μg.

2 μg of anti-CDK1 antibody (Santa Cruz Biotechnologies Inc.) and 20 μl of Sepharose beads (Bio-Rad Laboratories, Inc.) coated with protein A were added to the substance and left in the stationary state at 4° C. for 1 hour so that the CDK1 and the anti-CDK1 antibody were reacted with each other.

After the reaction, the beads were washed 3 times with a beads washing buffer (containing 0.1 w/v % Nonidet P-40 and 50 mM Tris-HCl, pH 7.0) and suspended again in 15 μl of the buffer A. As a result, a sample containing Sepharose beads having the CDK1 bound thereto via the anti-CDK1 antibody could be obtained.

10 μg of CDK1 substrate solution (containing 10 μg of histone H1 (Upstate Biotechnology Inc.), 5 mM ATP-γS (Sigma Corporation), 20 mM Tris-HCl, pH 7.4, and 0.1% Triton X-100) was added to the sample. The substrate solution was prepared and added to the tube such that the total amount of the mixed solution in the tube was 50 μl. The mixture was shaken at 37° C. for 10 minutes to induce the kinase reaction to introduce the monothiophosphoric acid group into histone H1.

After the kinase reaction, the reaction solution was centrifuged at 2000 rpm for 20 seconds to precipitate the beads. As a result, 18 μl supernatant was obtained.

15 μl of a binding buffer (containing 150 mM Tris-HCl, pH 9.2 and 5 mM EDTA) and 10 mM of an iodoacetylbiotin solution (containing 100 mM Tris-HCl, pH 7.5 and 1 mM EDTA) were added to the supernatant and left at room temperature in the dark for 90 minutes, thereby binding the iodoacetylbiotin to a sulfuric atom of the substrate (monothiophosphorylated substrate) into which the monothiophosphoric acid group had been introduced.

The reaction between the iodoacetylbiotin and the monothiophosphoric acid group was terminated by addition of 2-mercaptoethanol.

A sample containing 0.4 μg of the monothiophosphorylated substrate having iodoacetylbiotin bound thereto was blotted onto a PVDF membrane with a slot blotter.

The PVDF membrane was blocked with a solution containing 1 w/v % BSA, and streptavidin-FITC (Vector Laboratories Inc.) was added thereto and reacted at 37° C for 1 hour.

After the reaction, the PVDF membrane was washed 3 times with 50 mM of washing solution B.

After the washing, fluorescence analysis of the PVDF membrane was carried out by using a fluorescence image analyzer Molecular Imager FX (Bio-Rad Laboratories, Inc.). The activation value was calculated based on a calibration curve.

To prepare the calibration curve, a solution containing a protein at two concentrations (biotin-labeled immunoglobulin) was blotted onto a PVDF membrane and then labeled with FITC in the same manner as described above, and the fluorescence intensity of the protein was measured with the fluorescence image analyzer. Therefore, the activity of 1 U (unit) of measured CDK1 is a value indicative of fluorescence intensity equal to that where the protein is 1 ng.

The activation value of the CDK2 was measured in a manner similar to the measurement of the activation value of the CDK1 except for the use of the anti-CDK2 antibody (Santa Cruz Biotechnology Inc.).

### (4) Calculation of CDK Specific Activity

The CDK specific activity (mU/ng) was calculated from the CDK activation value and the CDK expression level measured above, in the following formula:

CDK specific activity=CDK activation value/CDK expression level

### (5) Establishment of Threshold Value

A second threshold value corresponding to the ratio between the CDK1 specific activity and the CDK2 specific activity (CDK2 specific activity/CDK1 specific activity) was established, and the result of comparison of the second threshold value with the CDK specific ratio, and the result of comparison of the comprehensive activity value of transmembrane tyrosine kinases in Example 1 with the threshold value (first threshold value) were combined to determine a risk of cancer relapse.

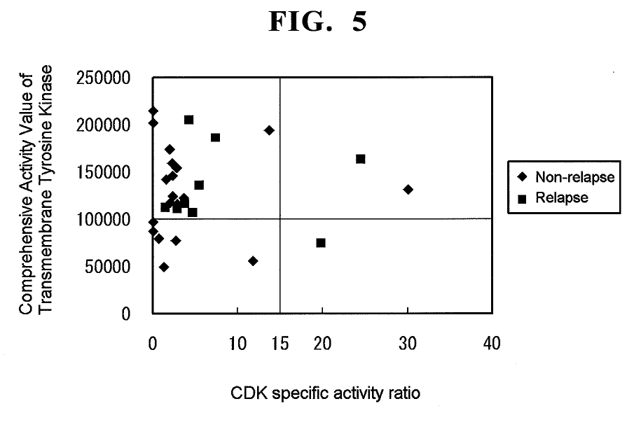

A graph was prepared by plotting determined comprehensive activity values of transmembrane tyrosine kinases from the 31 breast cancer patients on the ordinate against the CDK specific activity ratio (abscissa). The graph is shown in FIG. 5. The first threshold value was 100000, which was the same as in Example 1. The second threshold was set at 15. This value is a value that can separate the CDK specific activity ratio into a high-value group and a low-value group. When the comprehensive activity of transmembrane tyrosine kinases is lower than the first threshold value (100000) and simultaneously when the CDK specific activity ratio is lower than the second threshold value (15), the risk of relapse was determined to be low (Relapse Risk “Low”).

### (6) Determination of Relapse Risk

From the results shown in FIG. 5, 28 patients out of the 31 breast cancer patients had a CDK specific activity ratio lower than the second threshold value (15). Out of the 28 patients, 6 patients had a comprehensive activity value of transmembrane tyrosine kinase lower than the first threshold value (100000), and the 6 patients were determined to be “low” in the risk of relapse. Out of the 6 patients, there was no patient who had a relapse in 5 years after surgery.

From the foregoing, the result of comparison of the comprehensive activity value of transmembrane tyrosine kinases with the first threshold value is combined with the result of comparison of the CDK specific activity ratio as a CDK parameter with the second threshold value, whereby a group with a low risk of relapse can be classified and whether a relapse occurs or not can be accurately predicted.

The foregoing detailed description and examples have been provided by way of explanation and illustration, and are not intended to limit the scope of the appended claims. Many variations in the presently preferred embodiments will be obvious to one of ordinary skill in the art, and remain within the scope of the appended claims and their equivalents.

What is claimed is:

1. A method for determining a risk of cancer relapse comprising a step of determining the risk of cancer relapse on the basis of a comprehensive activity value of a transmembrane tyrosine kinase of a tumor cell.

2. The method according to claim 1, wherein the determining step is performed so as to compare the comprehensive activity value of a transmembrane tyrosine kinase with a threshold value, and determine the risk of cancer relapse is low when the activity value is lower than the threshold value.

3. The method according to claim 1, wherein the transmembrane tyrosine kinase includes at least two selected form insulin-like growth factor receptor (IGFR), platelet-derived growth factor receptor (PDGFR), human epithelial growth factor receptor (HER), and vascular endothelial growth factor receptor (VEGFR).

4. The method according to claim 1, wherein the comprehensive activity value of a transmembrane tyrosine kinase is obtained by following steps, comprising:
preparing a reagent including plural types of transmembrane tyrosine kinase;
phosphorylating a substrate of at least two types of transmembrane tyrosine kinase by contacting the transmembrane tyrosine kinase in the reagent with the substrate;
detecting the phosphorylated substrate; and
obtaining the comprehensive activity of the transmembrane tyrosine kinase in the reagent on the basis of a result of detecting step.

5. The method according to claim 4, wherein the preparing step is performed so as to prepare the reagent by crushing the tumor cell included in a tumor tissue in buffer solution, mixing the crushed tumor cell and a solution including a surfactant, centrifuging the mixture, and obtaining a supernatant after centrifugation.

6. The method according to claim 5, wherein the surfactant is nonionic surfactant.

7. The method according to claim 4, wherein the substrate is a mixture of plural types of substrates respectively having high specificity to a predetermined transmembrane tyrosine kinase.

8. The method according to claim 4, wherein the substrate is a universal substrate which can be a substrate of plural types of transmembrane tyrosine kinase.

9. A method for determining a risk of cancer relapse comprising a step of determining the risk of cancer relapse on the basis of a comprehensive activity value of a transmembrane tyrosine kinase of a tumor cell and a parameter of a cyclin-dependent kinase (CDK) obtained from an expression level and an activity value of CDK of a tumor cell.

10. The method according to claim 9, wherein the determining step is performed so as to compare the comprehensive activity value of a transmembrane tyrosine kinase with a first threshold value, compare the parameter of CDK with a second threshold value, and determine the risk of cancer relapse is low when the activity value is lower than the first threshold value and the parameter of CDK is lower than the second threshold value.

11. The method according to claim 9, wherein the transmembrane tyrosine kinase includes at least two selected form insulin-like growth factor receptor (IGFR), platelet-derived growth factor receptor (PDGFR), human epithelial growth factor receptor (HER), and vascular endothelial growth factor receptor (VEGFR).

12. The method according to claim 9, wherein the comprehensive activity value of a transmembrane tyrosine kinase is obtained by following steps:
a step of preparing a reagent including plural types of transmembrane tyrosine kinase;
a step of phosphorylating a substrate of at least two types of transmembrane tyrosine kinase by contacting the transmembrane tyrosine kinase in the reagent with the substrate;
a step of detecting the phosphorylated substrate; and
a step of obtaining the comprehensive activity of the transmembrane tyrosine kinase in the reagent on the basis of a result of detecting step.

13. The method according to claim 12, wherein the preparing step is performed so as to prepare the reagent by crushing the tumor cell included in a tumor tissue in buffer solution, mixing the crushed tumor cell and a solution including a surfactant, centrifuging the mixture, and obtaining a supernatant after centrifugation.

14. The method according to claim 13, wherein the surfactant is nonionic surfactant.

15. The method according to claim 12, wherein the substrate is a mixture of plural types of substrates respectively having high specificity to a predetermined transmembrane tyrosine kinase.

16. The method according to claim 12, wherein the substrate is a universal substrate which can be a substrate of plural types of transmembrane tyrosine kinase.

17. The method according to claim 9, where in the parameter of CDK is a CDK specific activity ratio which is a ratio between a first CDK specific activity and a second CDK specific activity,
wherein the first CDK specific activity is a ratio between an activity value and an expression level of the first CDK of the tumor cell, and
the second CDK specific activity is a ratio between an activity value and an expression level of the second CDK of the tumor cell.

18. The method according to claim 17, wherein the CDK specific activity ratio is obtained by following steps:
a step of measuring the activity values and the expression levels of the first CDK and the second CDK respectively;
a step of calculating the first CDK specific activity and the second CDK specific activity on the basis of a measuring result; and
a step of calculating the CDK specific activity ratio on the basis of the first CDK specific activity and the second CDK specific activity.

19. A computer program product for determining a risk of a cancer relapse, comprising:
a computer readable medium; and
instructions, on the computer readable medium, adapted to enable a general purpose computer to perform operations, comprising:
a step of comparing a comprehensive activity value of a transmembrane tyrosine kinase of a tumor cell with a threshold value; and
a step of determining the risk of cancer relapse on the basis of a comparison result of the comparing step.

20. The computer program product according to claim 19, wherein the comparing step further compares a parameter of a cyclin-dependent kinase (CDK) obtained from an expression level and an activity value of CDK of a tumor cell with a second threshold value.
